(19) United States
(12) Patent Application Publication
(10) Pub. No.: US 2015/0057618 A1
(43) Pub. Date: Feb. 26, 2015

(54) Long Term Implant Access Line Retention Device And Method Of Use

(71) Applicant: SEC MEDICAL DEVELOPMENT, INC., San Antonio, TX (US)

(72) Inventors: Abram Escobedo, San Antonio, TX (US); Oscar Cantu, San Antonio, TX (US); Aaron Dale Saul, JR., Harper, TX (US); David Wesley Stephens, Dallas, TX (US)

(21) Appl. No.: 14/465,802

(22) Filed: Aug. 21, 2014

Related U.S. Application Data
(60) Provisional application No. 61/887,428, filed on Oct. 6, 2013.
(60) Provisional application No. 61/868,496, filed on Aug. 21, 2013.

(51) Int. Cl. A61M 25/02; A61M 39/02

(52) U.S. Cl. CPC A61M 25/02; A61M 39/02; A61M 2025/0246; A61M 2025/0253; A61M 2039/0258; A61M 2039/0261
USPC 604/180

(57) ABSTRACT

Disclosed herein is an insertion site protection device that attaches to an access device. Specifically, the invention is a retention device for access lines and needles used for long-term access of drug delivery portals and pumps used to treat patients for a variety of diseases. The device will allow the clinician to visualize the skin at the insertion site for signs and symptoms of complications such as infection and adds an extra level of securement to help lessen the risk of inadvertent explant of the needle from the fully implanted access device. This is desired as the inadvertent removal of the needle may cause permanent damage to the access device, shortening the devices lifespan or necessitating device replacement as well as damage the skin and tissues over the device causing injury or infection to the patient and making the insertion site unusable.

### CROSS-REFERENCE TO RELATED APPLICATIONS

This application claims the benefit under Title 35 United States Code §119(e) of U.S. Provisional Patent Application Ser. No. 61/868,496; Filed: Aug. 21, 2013, and U.S. Provisional Patent Application Ser. No. 61/887,428; Filed: Oct. 6, 2013, the full disclosures of which are incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention generally relates to a device and method of use directed to an insertion site protection device used to attach an access. More specifically, the invention generally relates to a retention device for access lines and needles (hereafter called access lines) used for ling term access of drug delivery portals and pumps used to treat patients for a variety of diseases.

### BACKGROUND OF THE INVENTION

Without limiting the scope of the disclosed device and method, the background is described in connection with a novel system and approach directed to a long term implant access line retention device and method of use.

It is common in the treatment of patients to utilize catheters, drug delivery portals, and pumps to introduce fluids and medications directly into the patient or to withdraw fluids from the patient. Often, it becomes desirable to maintain such access over an extended period of time during the treatment of a patient. In order to keep the access line properly positioned for the duration of the treatment, the access line can be secured to the patient in a variety of ways. Most commonly, the primary retention method involves taping the access line to the patient.

Securing an access line with tape upon the patient has certain drawbacks. For one, the tape is ineffective at preventing rotation of the access line. Also, the use of tape as the primary retention method at the insertion site can retain dirt or other contaminant particles, potentially leading to infection of the patient. The tape often blocks the insertion site from inspection and cleaning, leading to poor or insufficient maintenance. Additionally, removal of taped dressings can cause undesired motion of the access lines, and irritation of the insertion site.

Taped dressings also require periodic changes. The frequent, often daily, removal and reapplication of adhesive tape to the skin of the patient can excoriate the skin area around the dressing. Such repeated applications of tape over the catheter or access line can additionally lead to the build-up of adhesive residue on the outer surface of the catheter or access line. This residue can also make the access line stickier and more difficult to handle for medical attendants.

While the primary approach may fulfill its unique purpose, it does not fulfill the need for a practical and effective means for providing a long term implant access line retention device where the access line can remain in place over an extended period of time, remain safe from unintended motion, and accomplish this without using tape as the primary retention method.

The present invention therefore proposes a novel system and method of use for securing retention lines which addresses the aforementioned drawbacks found in the prior art.

### BRIEF SUMMARY OF THE INVENTION

The present invention, therefore, provides a device and method of use for a long term implant access line retention device and method of use.

In one embodiment, the device is comprised of at least one anchor pad including a lower adhesive surface configured to attach to a patient's skin with a circular opening positioned therein, a plurality of ties and latches for access line retention, and a retainer for the access line tubing to be therein constrained. The shape of the device is preferentially rectangular with a plurality of legs that achieve the securement through means of the lower adhesive surface, certain portions of which are specifically non-adhesive to prevent excessive attachment to the patient. In another embodiment, the device is made from materials that allow for the site to be fully viewable through utilization of clear materials such as silicone. In yet another embodiment, the device is also comprised of an antimicrobial coating or material or the means to interface with existing antimicrobials (such as Chlorhexidine) or antimicrobial patches in order to protect the insertion site from infection. In another embodiment the device is also comprised of an elastomeric belt preferentially shaped to accommodate a large variety of infusion set styles through the compliance of the device. In another embodiment, the device is further comprised of an infusion set as an integrated feature with a needle stick prevention feature upon removal. In yet another embodiment the device involves an access line retention system that includes either the use of an elastomeric belt preferentially shaped to accommodate a large variety of infusion set styles through the compliance of the retention system. In an additional embodiment the invention involves a an access line retention system in which the infusion set is an integrated feature of the device, and the retention device features form a needle stick prevention feature upon removal. The invention may also be used to stabilize needles in access sites during hemodialysis.

In summary, the present invention generally relates to a device and method of use directed to a long-term implant access line retention device. More specifically, the invention generally relates to a retention device for access lines and needles (hereafter called access lines) used for ling term access of drug delivery portals and pumps used to treat patients for a variety of diseases.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is an illustration of a drug delivery portal in accordance with embodiments of the disclosure;

FIG. 2 is a side illustration of a port placement beneath the skin being accessed by a needle in accordance with embodiments of the disclosure;

FIG. 3 is a side view of an infusion set used for accessing ports and other long term implant devices illustrating the extension tubing, the luer, winged hub, and the huber needle in accordance with embodiments of the disclosure;

FIG. 4 is a top view of the long term implant access line retention device in accordance with embodiments of the disclosure;

FIG. 5 is a side view of the retention clip and belt of the long term implant access line retention device in accordance with embodiments of the disclosure;

FIG. 6 is a side view of the tubing clamp of the long term implant access line retention device in accordance with embodiments of the disclosure;

FIG. 7 is a top view of the long term implant access line retention device illustrating an infusion set in place via the retention clips and belts with the infusion set tubing in the tubing clamp in accordance with embodiments of the disclosure;

FIG. 8 is a side view of the of the long term implant access line retention device illustrating an infusion set in place via the retention clips and belts with the infusion set tubing in the tubing clamp and the port under the skin in accordance with embodiments of the disclosure;

FIG. 9 is an isometric view of the long term implant access line retention device illustrating the infusion set and elastomeric belt retained by locking clips. In accordance with embodiments of the disclosure;

FIG. 10 is a front view of the long term implant access line retention device illustrating the infusion set, elastomeric belt, and elastomeric belt retention locking clips in accordance with embodiments of the disclosure;

FIG. 11 is an isometric view of the long term implant access line retention device illustrating the elastomeric belt, elastomeric belt retention locking clip, thumb tab, locking clip, and infusion set tubing notch in accordance with embodiments of the disclosure;

FIG. 12 is an isometric view of the long term implant access line retention device illustrating an integrated infusion set with an adhesive substrate attached to the skin of the user in accordance with embodiments of the disclosure;

FIG. 13 is a side view of the long term implant access line retention device illustrating the integrated infusion with an adhesive substrate attached to the skin of the individual in accordance with embodiments of the disclosure;

FIG. 14 is an isometric view of the long term implant access line retention device illustrating the integrated infusion set with an adhesive substrate in accordance with embodiments of the disclosure;

FIG. 15 is an isometric view of the long term implant access line retention device illustrating the integrated infusion set with an adhesive layer and a frame in accordance with embodiments of the disclosure;

FIG. 16 is a front view of the long term implant access line retention device illustrating the infusion set with an elastomeric belt retained by locking clips in accordance with embodiments of the disclosure;

FIG. 17 is an isometric view of the long term implant access line retention device illustrating the belt retention clip, thumb tab, locking clip, and infusion set tubing clamp in accordance with embodiments of the disclosure;

FIG. 18 is an isometric view of the long term implant access line retention device illustrated with an integrated infusion set attached directly to the adhesive substrate with backer tab in accordance with embodiments of the disclosure;

FIG. 19 is side view of the long term implant access line retention device illustrated with an integrated infusion set attached directly to the adhesive substrate with backer tab in accordance with embodiments of the disclosure;

FIG. 20 is an isometric view of the long term implant access line retention device illustrated with an integrated infusion set attached directly to the adhesive substrate after removal in accordance with embodiments of the disclosure;

FIG. 21 is a top view of the long term implant access line retention device illustrated with an integrated infusion set and an adhesive substrate in accordance with embodiments of the disclosure;

FIG. 22 is a side view of the long term implant access line retention device illustrated with an integrated infusion set and an adhesive substrate in accordance with embodiments of disclosure;

FIG. 23 is an isometric view of the long term implant access line retention device illustrated with an integrated infusion set and an adhesive substrate.

### DETAILED DESCRIPTION OF THE INVENTION

Disclosed herein is an improved device and method of use for a long term implant access line retention device. The numerous innovative teachings of the present invention will be described with particular reference to several embodiments (by way of example, and not of limitation).

Reference is first made to FIG. 1, an illustration of a drug delivery portal in accordance with embodiments of the disclosure. Shown here in an anatomical fashion is the position of the long term implanted medication port 1 in the patient's upper right chest under the clavicle. The long term implant access line retention device is meant to be placed on the patient's skin over the long term implanted medication port so that the needle used to access the device can be secured to the patient as to limit accidental removal of the needle causing trauma both to the patient and the long term implanted medication port septum. FIG. 1 illustrates a drug delivery portal 1 also called a port showing a typical sub-clavian vein 2 placement with associated anatomy. This is provided for clarification of one type of access the subject device is expected to be used for.

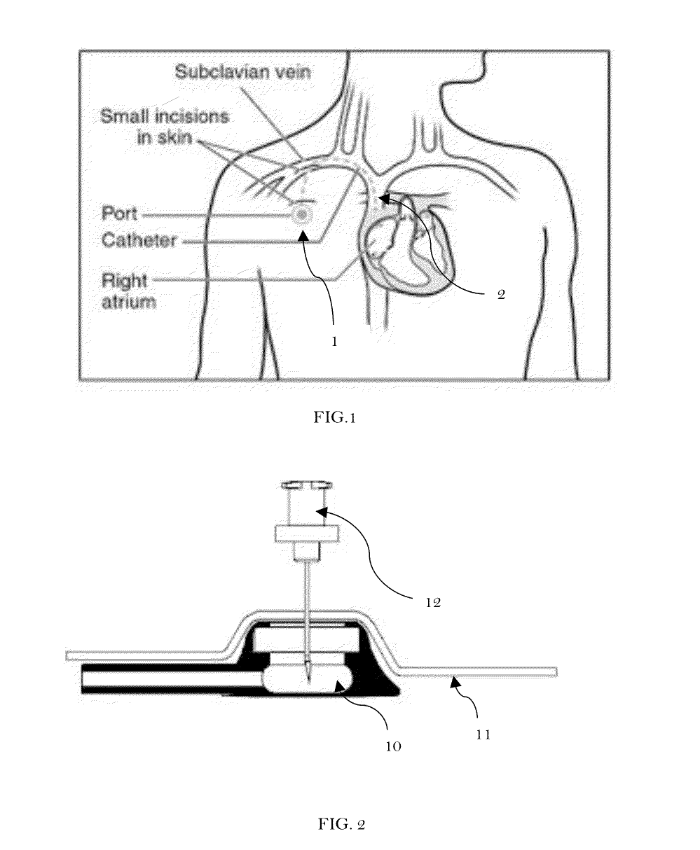

Reference is next made to FIG. 2, a side illustration of a port placement beneath the skin 11 being accessed by a needle 12 in accordance with embodiments of the disclosure. Shown here is how the long term implanted medication port 10 is accessed with a needle 12 through the patient's skin 11. The needle 12 is inserted through the patient's skin 11 into the septum of the long term implanted medication port 10 with the needle 12 tip residing in the reservoir of the long term implanted medication port 10.

Reference is now made to FIG. 3, a side view of an infusion set used for accessing ports and other long term implant devices illustrating the extension tubing 21, the luer 22, winged hub 23, and the huber needle 24 in accordance with embodiments of the disclosure. Seen here is a depiction of an access needle 24 and it's tubing extension 21 that is used to access the long term implanted medication port. The needle 24 is what is termed to be a “non-coring needle” with an offset bevel. The access device is controlled by holding the device by the “head” 23 which allows the accessing clinician to control the insertion of the needle into the long term implanted medication port. The clamp is engaged during the period of time when medications or fluids are not being infused through the device and is released or “opened” when medications and/or fluids are being infused into the patient through the long term implanted medication port. The luer connection 22 is where the clinician connects the tubing that is attached or inserted into a bag of medication, or in some cases a syringe of medication, that will be administered to the patient through the long term implanted medication device.

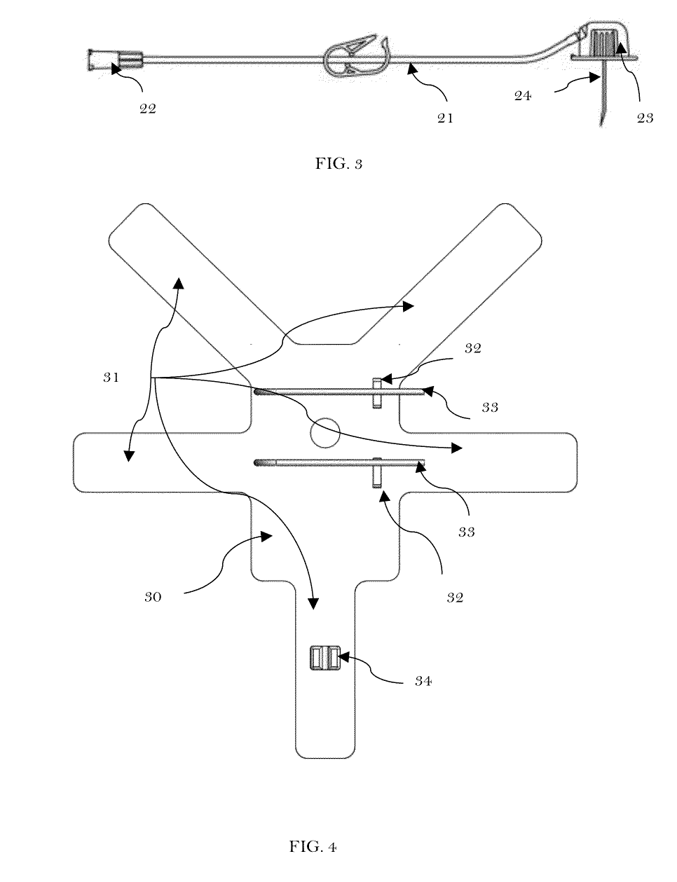

Reference is now made to FIG. 4, a top view of the long-term implant access line retention device in accordance with embodiments of the disclosure. In this depiction you will note the presence of five (5) “legs” 31 as part of the device's body element 30 that is placed over the implant device in the patient after cleansing the skin with the intent to sterilize the area for insertion of a needle through the long term implant access retention device into the long term implanted medication port present in the patient's chest. The legs portion of the body 31 and/or the central body portion 30 attaches to the patient by an attachment means such as having an adhesive on the lower portion or sides of the legs 31 and/or central body portion 30 in contact with the patient. Once inserted into the long-term implanted medication port, the needle assembly is secured by pulling over the device the tie down or belts 33 (two illustrated in this figure) and securing it into the locking assembly 32 (two illustrated in this figure) thus securing the needle to the patient's chest. That is the tie down or belts 33 is an attachment element providing a means for securing the needle assembly to the patient by locking the tie down or belt element 33 into the locking element 32 or may be referred to as a locking assembly 32 which are also referred to and may be as an example and not a limitation, a tie down, arm, belt retention clamps. In an embodiment, as illustrated, the arms 33 rotate about their proximal end (connecting portion which is opposite of the end which is held down by the locking assembly 32). The tubing of the needle assembly is then locked into the “tail” of the device via the tubing retention clamp 34. The body element 30 also has an opening portion to view the skin at the insertion site for signs and symptoms of complications such as infection as well as allow access to site for a needle as an example. In this illustration, the opening portion is approximately in the center of the body element 30 between the two attachment elements 33 and on the left side of the two locking elements 32. In another embodiment, the opening portion is provided by the shape of the body element 30 and is not within the interior portion of the body element 30. That is the body element 30 is shaped so that the body element 30 does not cover the access site. This will be discussed in more detail with the following figures.

Reference is now made to FIG. 5, a side view of the retention clamp or clip (locking element) 40 and a tie down or belt (attachment element) 41 of the long-term implant access line retention device in accordance with embodiments of the disclosure. Seen here are the locking mechanism that ensures the access needle is secured in place. The tie down or belt 41 is attached to the opposite side of the center section of the long-term implant access retention device. The locking portion of the tie down or belt 40 is positioned on the opposite side of the long term implant access retention device so that when the tie down or belt 41 is passed over the needle assembly is can be secured in place with the retention clamps or clips 40.

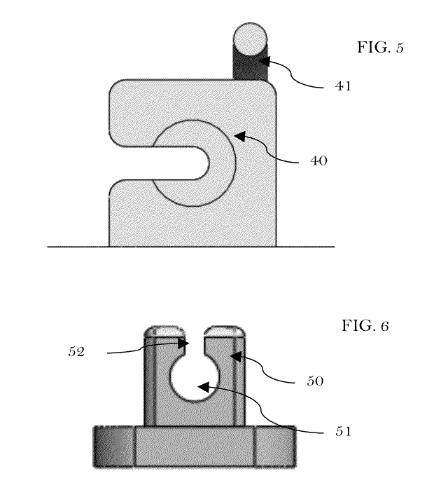

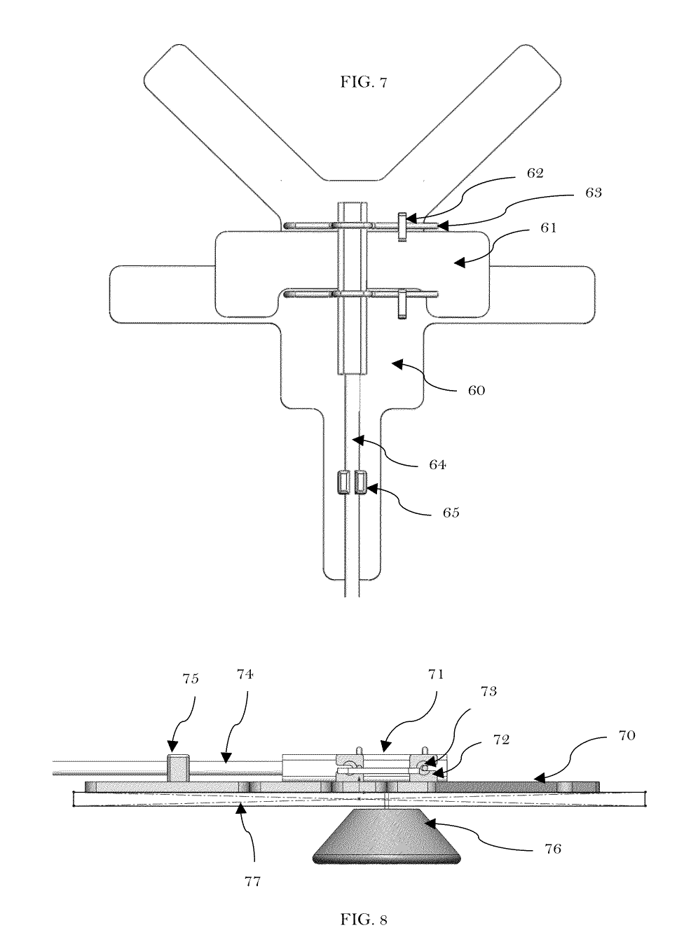

Reference is now made to FIG. 6, a side view of the tubing retention clamp 50 of the long-term implant access line retention device in accordance with embodiments of the disclosure. Here seen is the tubing retention clamp 50 and the tubing hole 51. The tubing of the needle assembly is passed through the gap 52 that is sized just smaller than the outer diameter of the tubing on the needle assembly to ensure securement in the tubing well or hole 51 of the tubing retention clamp 50.

Reference is now made to FIG. 7, a top view of the long term implant access line retention device illustrating an infusion set in place via the locking assembly 62 and belts or tie downs 63 with the infusion set tubing 64 in the tubing retention clamp 65 in accordance with embodiments of the disclosure. Here seen is the long-term implant access line retention device deployed. The needle assembly wing 61 is seen in the flat position post insertion of the needle assembly into the long-term implanted medication port. The belts or tie down 63 has passed over the needle assembly and is locked into the locking portion of the tie down mechanism or locking assembly 62. The extension tubing of access needle assembly 64 can be seen secured into the tubing retention clamp 65.

Reference is now made to FIG. 8, a right side view of the of the long term implant access line retention device illustrating an infusion set in place via the tie down and belt retention clips 72 and belts or tie downs 73 with the infusion set tubing 74 in the tubing retention clamp 75 and the port 76 under the skin 77 in accordance with embodiments of the disclosure. Here seen is a side view of the long-term implant access line retention device deployed through the patients skin 77 into the long term implanted medication port 76. Again note the access needle assembly 73 with the tie down pulled over the access needle assembly and locked into the locking portion of the tie down mechanism 72. Again note the tubing of the access needle assembly 75 has been inserted into the tubing retention clamp 74.

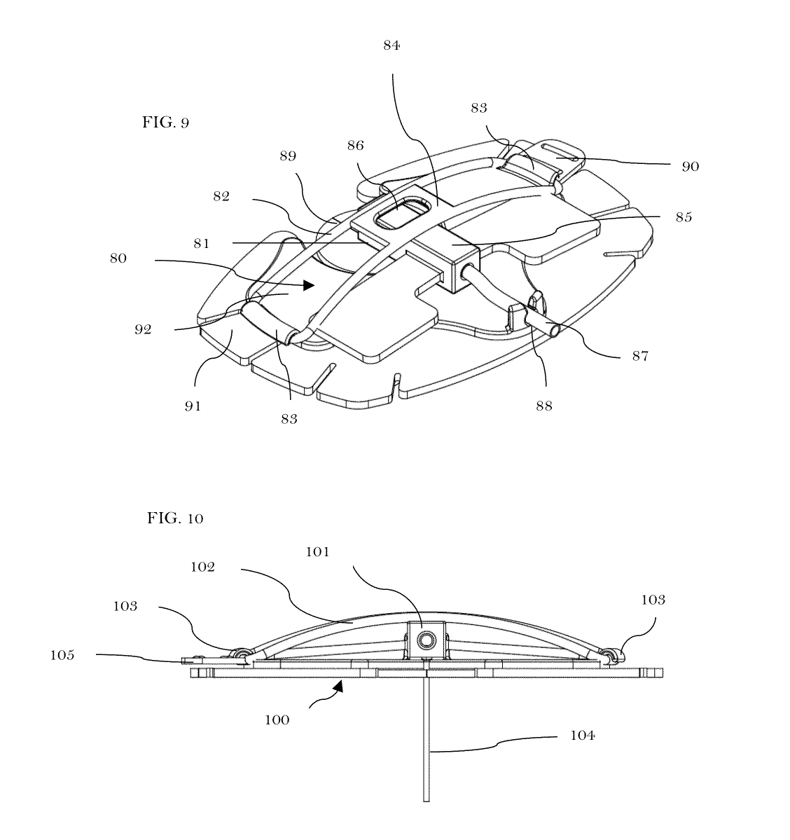

Reference is now made to FIG. 9, an isometric top view of the long term implant access line retention device illustrating the infusion set and elastomeric belt retained by locking clips. in accordance with embodiments of the disclosure. Illustrated in this view of the long term implant access line retention device 80 includes and adhesive layer 91 and a frame 92 which are permanently attached. The adhesive layer includes finger elements or leg portions 91 (finger like features) which allow for the adhesive to conform to a patient's anatomy while the frame allows for the infusion set to be rigidly constrained. The infusion set 81 is shown in place locked down using an elastomeric belt 82. The belt is designed to restrain the infusion set, and prevent unintentional displacement stretching across the infusion set, with enough flexibility to accommodate the large variety of infusion set styles. The belt 82 is retained by locking clips 83, and the belt 82 is substantially ladder shaped with a central feature 84 designed to conform to the infusion sets hub shape 85 and a finger tab feature 90 that allows for the band to be stretched into position and locked down. The central shape includes a window 86 designed to allow visualization of the insertion site for inspection and cleaning. Said window may take on various shapes such as but not limited to circular, oval, obloid, or rectangular. Also included is a circular window opening shape 89 which has also been referred to as the body element's opening portion around the site that is preferentially sized and shaped to provide for direct site visualization and an area for placement of an antimicrobial patch. The infusion set tubing 87 is restrained in the tubing retention clamp 88 to prevent unintentional rotation of the infusion set.

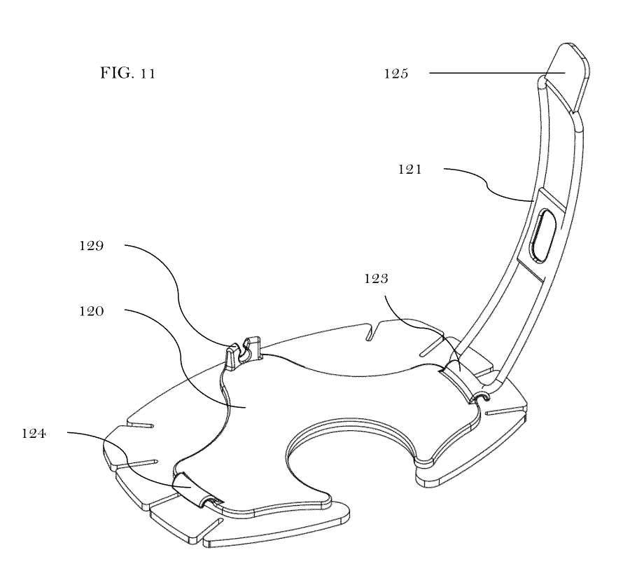

Reference is now made to FIG. 10, a front view of the long term implant access line retention device illustrating the infusion set 101, elastomeric belt 102, and elastomeric belt retention locking clips 103 in accordance with embodiments of the disclosure. In this view of the long term implant access line retention device 100, the relationship of the infusion set 101 can be seen as it is expected to be in place and locked down by the elastomeric belt 102. The belt 102 is shown stretched over and retained by locking clips 103. One side of the belt in an embodiment is preferentially permanently attached. Also shown is the needle 104 which would be implanted through the skin and in a drug delivery port or similar device. The finger tab 105 is shown in the locked down position, laying flat against the adhesive layer. A user of the device would be able to pull up on the finger tab 105.

Reference is now made to FIG. 11, an isometric top view from the front of the long term implant access line retention device illustrating the elastomeric belt 121, elastomeric belt retention locking clips 123,124, thumb tab 125, and infusion set tubing retention clip 129 in accordance with embodiments of the disclosure. Illustrated here is the long term implant access line retention device 120 with no infusion set in place as it would be prior to placement. The elastomeric belt 121 is shown in the unlocked position with the primary locking clips 123 shown as the band is positioned when open, and the other locking clip 124 can be seen. The belt will be pulled into position using the thumb tab 125 into locking clip 124 to retain the infusion set. Also shown is the infusion set tubing retention clamp 129 which prevents rotational movement.

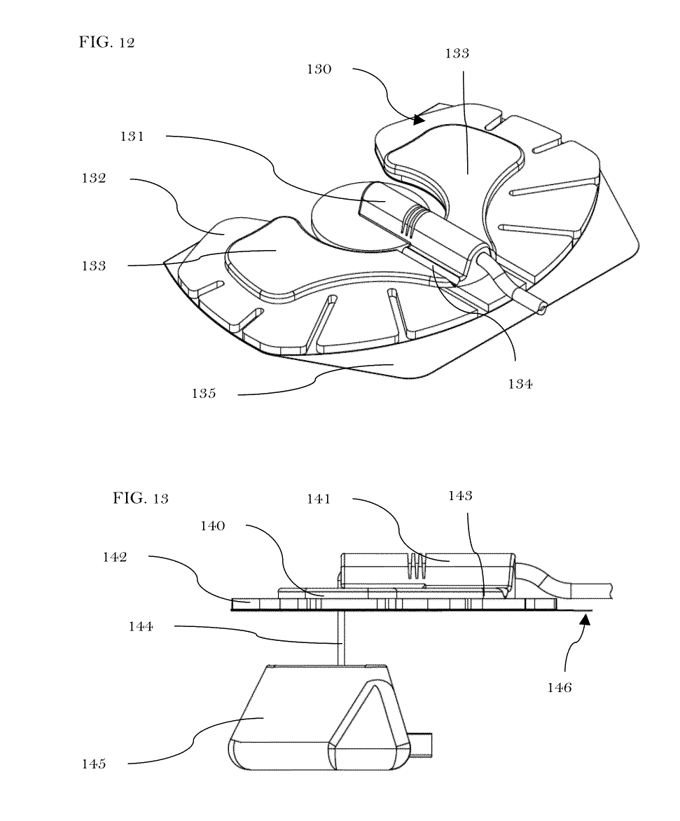

Reference is now made to FIG. 12, an isometric view of the long term implant access line retention device illustrating an integrated infusion set with an adhesive substrate attached to the skin of the user in accordance with embodiments of the disclosure. Illustrated here is the long term implant access line retention device 130 with an integrated infusion set 131 attached directly to the adhesive substrate 132 stuck to the skin 135 such that the infusion set wings 133 include hinging elements 134 or may be referred to as living hinges 134 designed to allow the infusion set wings 133 to fold over the needle when the infusion set 131 is removed. The adhesive substrate will then stick the two wings 133 together, making the needle inaccessible, and thus safe from unintentional needle stick.

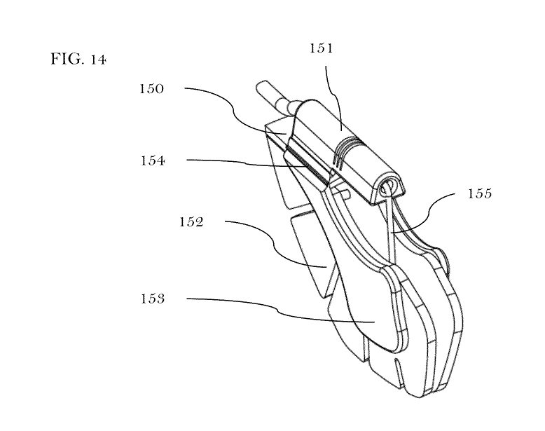

Reference is now made to FIG. 13, a side view of the long term implant access line retention device illustrating the integrated infusion with an adhesive substrate attached to the skin of the individual in accordance with embodiments of the disclosure. Illustrated here is the long term implant access line retention device 140 with an integrated infusion set 141 attached directly to the adhesive substrate 142 stuck to the skin 146 such that the infusion set wings 143 are designed to allow the infusion set wings to fold over the needle 144 when the infusion set is removed. The drug delivery port 145 is shown with the needle 144 in place.

Reference is now made to FIG. 14, an isometric view of the long term implant access line retention device illustrating the integrated infusion set with an adhesive substrate in accordance with embodiments of the disclosure. Illustrated here is the long term implant access line retention device 150 with an integrated infusion set 151 attached directly to the adhesive substrate 152 such that the infusion set wings 153 include living hinges 154 designed to allow the infusion set wings to fold over the needle 155 when the infusion set is removed. The device is shown in its closed position with the wings 153 folded and covering the needle 155 and the adhesive substrate 152 sticking the two wings 153 together, making the needle inaccessible 155, and thus safe from unintentional needle stick.

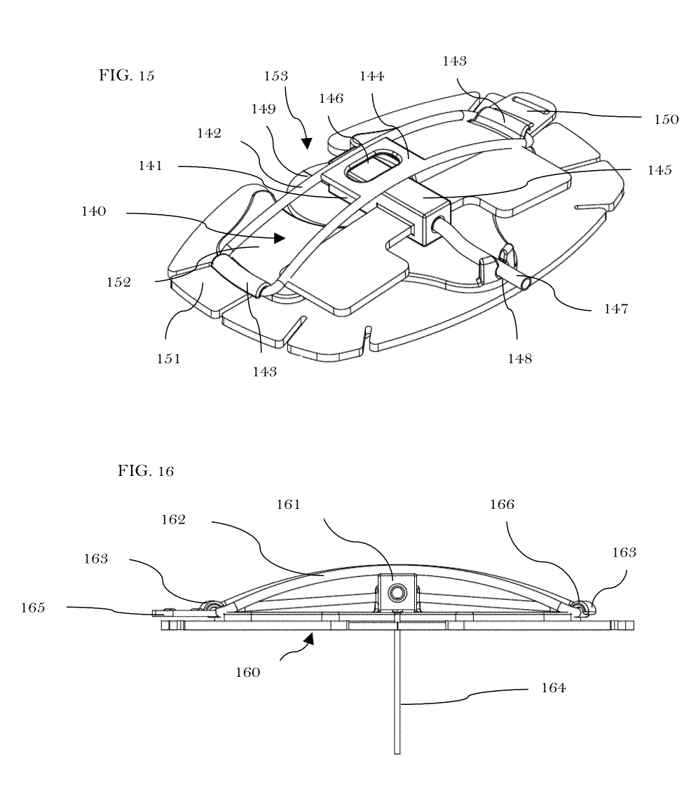

Reference is now made to FIG. 15 an isometric view of the long term implant access line retention device 140 which includes and adhesive layer 151 and a frame 152 which are permanently attached. The adhesive layer includes finger like features which allow for the adhesive to conform to a patient's anatomy while the frame 152 allows for the infusion set 141 to be rigidly constrained. The infusion set 141 is shown in place locked down using an elastomeric belt 142. The belt is designed to restrain the infusion set, and prevent unintentional displacement stretching across the infusion set, with enough flexibility to accommodate the large variety of infusion set styles. The belt 142 can alternately be a cable tie, plastic clip, hook and loop, or other similar securement method. The belt is retained by locking clips 143, and is substantially ladder shaped with a central feature 144 designed to conform to the infusion sets hub shape 145 and a finger tab feature 150 that allows for the band to be stretched into position and locked down. The central shape includes a window 146 designed to allow visualization of the insertion site for inspection and cleaning. Said window may be circular, oval, obloid, or rectangular. Also included is a semi-circular window 149 around the site that is preferentially sized and shaped to provide for direct site visualization and an area for placement of an antimicrobial patch. The opening 153 in the semi-circular window allows for passage of the device into place after the needle has been inserted. The infusion set tubing 147 is restrained in the tubing retention clamp 148 to prevent unintentional rotation of the infusion set 141.

Reference is now made to FIG. 16 a front view of the long term implant access line retention device 160 wherein the relationship of the infusion set 161 can be seen as it is expected to be in place and locked down by the elastomeric belt 162. The belt 162 is shown stretched over the infusion set 161 and retained by locking clips 163. In an embodiment, one side of the belt 162 is preferentially permanently attached at bond point 166. Also shown is the needle 164 which would be implanted through the skin and in a drug delivery port or similar device. The finger tab 165 is shown as it will be in the locked down position, laying flat against the adhesive layer.

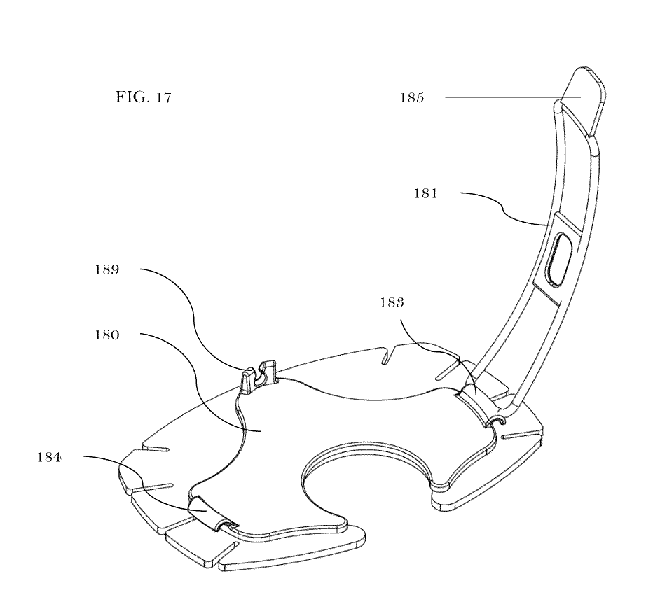

Reference is now made to FIG. 17 you can see an isometric view of the long term implant access line retention device 180 with no infusion set in place as it would be prior to placement. The elastomeric belt 181 is shown in the unlocked position with the primary locking clips 183 shown as the band or belt 181 is positioned when open, and the other locking clip 184 can be seen. The belt 181 will be pulled into position using the thumb tab 185 into locking clip 184 to retain the infusion set. Also shown is the infusion set tubing retention clamp 189 which prevents rotational movement.

Reference is now made to FIG. 18 an isometric view of the long term implant access line retention device 190 with an integrated infusion set 191 attached directly to the adhesive substrate 192 and attached to a backer 195 with a tab to allow for removal of said backer after insertion of needle. The infusion set 191 incorporates flexible joint 196 allowing for flexing of the needle portion to accommodate varying depths of delivery port. The infusion set wings 193 include living hinges 194 designed to allow the infusion set wings to fold over the needle when the infusion set 191 is removed. The adhesive substrate 192 will then stick the two wings 193 together, making the needle inaccessible, and thus safe from unintentional needle stick. The wings 193 can also be locked by means of a clip or stay to retain the two wings together.

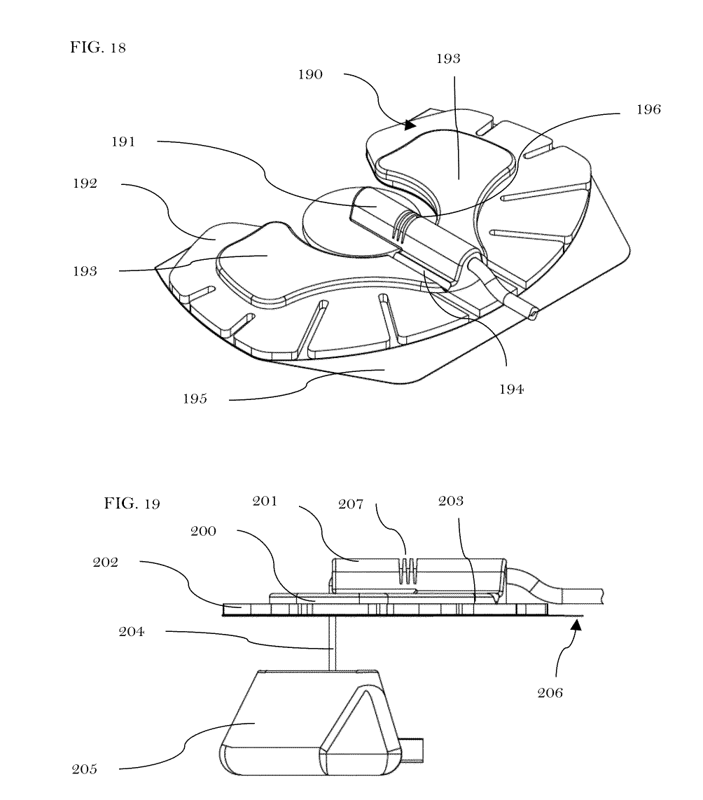

Reference is now made to FIG. 19 a side view of the long term implant access line retention device 200 with an integrated infusion set 201 attached directly to the adhesive substrate 202 and attached to a backer 206 with a tab to allow for removal of said backer after insertion of needle. The infusion set wings 203 are designed to allow folding over the needle 204 when the infusion set 201 is removed. The drug delivery port 205 is shown with the needle 204 in place. The infusion set 201 incorporates flexible joint 206 allowing for flexing of the needle 204 portion to accommodate varying depths of delivery port. The infusion set 201 incorporates flexible joint 206 allowing for flexing of the needle portion 204 to accommodate varying depths of delivery port 205.

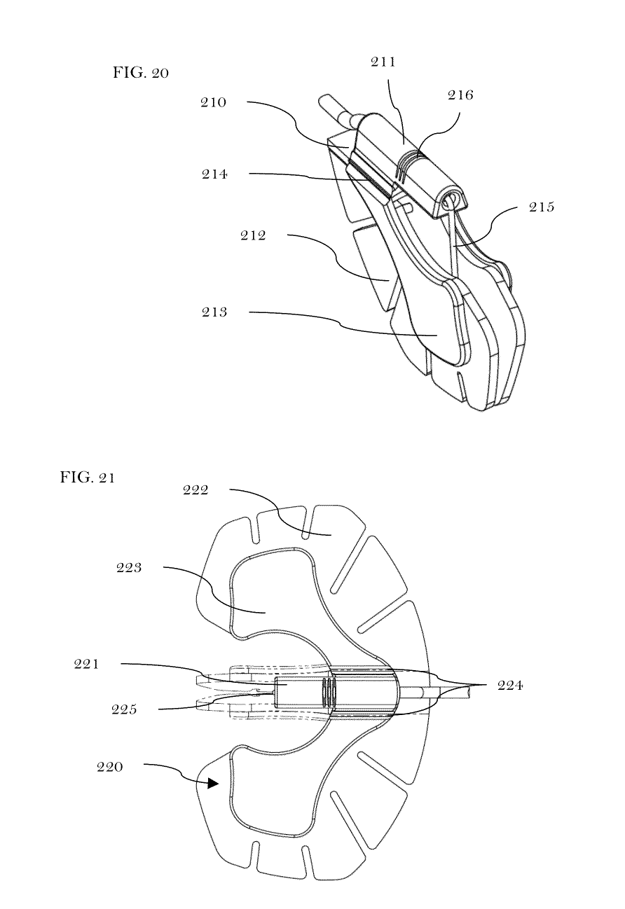

Reference is now made to FIG. 20 an isometric view of the long term implant access line retention device 210 with an integrated infusion set 211 attached directly to the adhesive substrate 212. The infusion set wings 213 include living hinges 214 designed to allow the infusion set wings 213 to fold over the needle 215 when the infusion set 211 is removed. The device is shown in its closed position with the wings 213 covering the needle and the adhesive substrate 212 sticking the two wings 213 together, making the needle 215 inaccessible, and thus safe from unintentional needle stick. The infusion set 211 incorporates flexible joint 216 allowing for flexing of the needle portion 215 to accommodate varying depths of delivery port.

Reference is now made to FIG. 21 a top view of the long term implant access line retention device 220 with an integrated infusion set 221 attached directly to the adhesive substrate 222 such that the infusion set wings 223 include living hinges 224 designed to allow the infusion set wings 223 to fold over the needle when the infusion set 221 is removed. The adhesive layer 222 and wings 223 are also shown in phantom lines (dashed) such that the device can be seen in its open and closed positions. The phantom lines illustrate the closed or folded position.

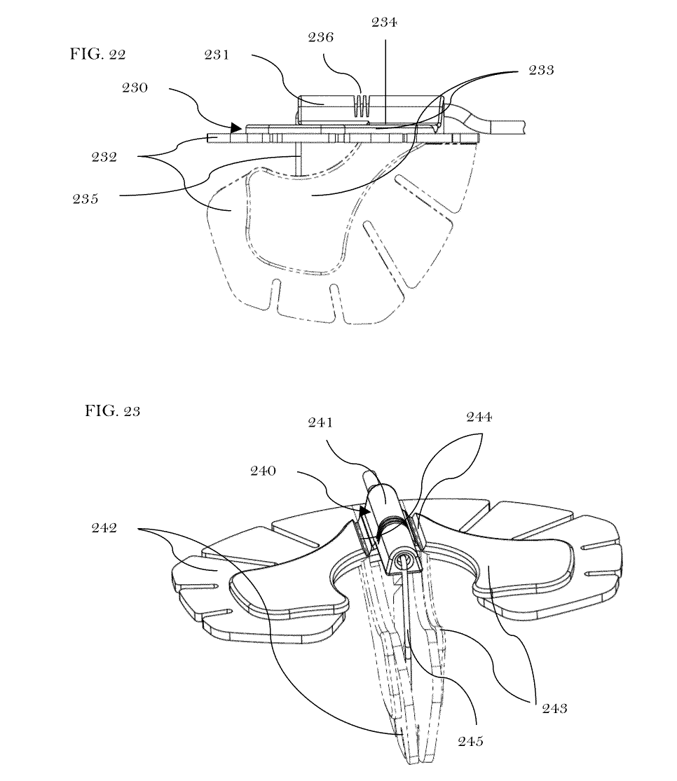

Reference is now made to FIG. 22 a side view of the long term implant access line retention device 230 with an integrated infusion set 231 attached directly to the adhesive substrate 232 such that the infusion set wings 233 include living hinges 234 designed to allow the infusion set wings 233 to fold over the needle 235 when the infusion set 231 is removed. The adhesive layer 232 and wings 233 are also shown in phantom lines (dashed) such that the device can be seen in its open and closed positions. The infusion set 231 incorporates flexible joint 236 allowing for flexing of the needle portion to accommodate varying depths of delivery port. The phantom lines illustrate the closed or folded position.

Reference is now made to FIG. 23 an isometric front view of the long term implant access line retention device 240 with an integrated infusion set 241 attached directly to the adhesive substrate 242 such that the infusion set wings 243 include living hinges 244 designed to allow the infusion set wings 243 to fold over the needle 245 when the infusion set 241 is removed. The adhesive layer 242 and wings 243 are also shown in phantom lines (dashed) such that the device can be seen in its open and closed positions. The phantom lines illustrate the closed or folded position.

In brief, as described herein provides for an effective and efficient device and method of use for a long-term implant access line retention device.

The disclosed device and method of use is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention.

Terms such as “a”, “an”, and “the” are not intended to refer to only a singular entity, but include the general class of which a specific example may be used for illustration. The terminology herein is used to describe specific embodiments of the invention, but their usage does not delimit the disclosed device or method, except as may be outlined in the claims.

Any embodiments comprising a one component or a multi-component device having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific device and method of use described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The device and/or methods disclosed and claimed herein can be made and executed without undue experimentation in light of the present disclosure. While the device and methods of this invention have been described in terms of preferred embodiments, it will be apparent to those skilled in the art that variations may be applied to the device and/or methods and in the steps or in the sequence of steps of the method described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

What is claimed is:

1. An access line retention device comprising:
a body element having an adhesive portion for attaching said device to a patient and said body element having an opening portion for accessing and viewing the skin at the insertion site;
an attachment element located on the top side of said device for securing a needle assembly to the device;
a locking element located on the top side of said device for holding into a closed position said attachment element.

2. The device of claim 1, further comprising a tubing retention clamp.

3. The device of claim 2, wherein said adhesive portion is on the bottom side of said body element.

4. The device of claim 2, wherein said body element is further comprised of at least one leg portion.

5. The device of claim 4, wherein the adhesive is on the bottom side of said body element's leg portion.

6. The device of claim 2, wherein said attachment element is an arm.

7. The device of claim 2, wherein said attachment element is an elastomeric belt.

8. The device of claim 2, wherein said attachment element is a tie down.

9. The device of claim 2, wherein said body element is made out of transparent material.

10. The device of claim 2, wherein said body element is further comprised of a frame to further secure the infusion set.

11. The device of claim 1, further comprising a tubing retention clamp; wherein said body element is further comprised of at least one leg portion; wherein the adhesive is on the bottom side of said body element's leg portion; wherein said body element is further comprised of a frame to further secure the infusion set; and wherein said body element is made out of transparent material.

12. An access line retention device comprising:
a body element having an adhesive portion for attaching said device to a patient and said body element having an opening portion for accessing and viewing the skin at the insertion site;
said body element having an integrated infusion set element;

13. The device of claim 12, further comprising a tubing retention clamp.

14. The device of claim 12, wherein said adhesive portion is on the bottom side of said body element.

15. The device of claim 12, wherein said body element is further comprised of at least one leg portion.

16. The device of claim 12, wherein said body element is made out of transparent material.

17. The device of claim 12, wherein said body element is further comprised of at least one hinging element to allows said body element to fold over the needle when the infusion set is removed form the patient.

18. The device of claim 12, wherein said body element is further comprised of two hinging elements to allows said body element to fold over the needle when the infusion set is removed form the patient.

19. The device of claim 12, wherein said integrated infusion set element further comprises a flexible joint.

20. The device of claim 12, further comprising a tubing retention clamp; wherein said adhesive portion is on the bottom side of said body element; wherein said body element is further comprised of at least one leg portion; wherein said body element is further comprised of two hinging elements to allows said body element to fold over the needle when the infusion set is removed form the patient; and wherein said integrated infusion set element further comprises a flexible joint.
